(19) United States
(12) Patent Application Publication
(10) Pub. No.: US 2024/0409578 A1
(43) Pub. Date: Dec. 12, 2024

(54) SOLID-PHASE SUPPORTED RADIOLABELING OF PEPTIDES

(71) Applicant: UNIVERSITY OF COPENHAGEN, Copenhagen K (DK)

(72) Inventors: Kristian STRØMGAARD, Roskilde (DK); Matthias Manfred HERTH, Malmö (SE); Andreas KJÆR, Frederiksberg (DK); Eduardo Felipe Alves FERNANDES, Copenhagen S (DK); Line Bruhn Schneider KNUDSEN, Gentofte (DK)

(21) Appl. No.: 18/273,957

(22) PCT Filed: Jan. 26, 2022
(86) PCT No.: PCT/EP2022/051665

§ 371 Date: Jul. 24, 2023

(30) Foreign Application Priority Data
Jan. 26, 2021 (EP) 21153498.7

(51) Int. Cl. C07K 1/13

(52) U.S. Cl. CPC C07K 1/13

(57) ABSTRACT

The invention regards a method for ¹⁸F-labeling of a peptide.

### TECHNICAL FIELD

The invention relates to ¹⁸F-labeling of peptides supported on a solid-support.

### BACKGROUND

Recent advances that allow de novo discovery of peptides including phage- or ribosome display may result in faster hit-to-lead optimization of peptides than traditional small-molecule development programs. Positron emission tomography (PET) is a widely used imaging modality for disease diagnosis, treatment monitoring and therapy planning where a high selectivity profile and a low non-displaceable binding component of a tracer are needed for successful imaging applications. Peptides can effectively be developed with these properties and as such, they are an interesting platform to facilitate the development of molecular imaging agents from bench-to-bedside. Fluorine-18 (¹⁸F) is the most frequently employed radionuclide in PET imaging as it possesses almost ideal nuclear decay characteristics for molecular imaging. High resolution, good dosimetry and scalability are just some of the advantages that this nuclide offers. However, the incorporation of fluorine-18 into peptides is often troublesome as clinically most relevant direct nucleophilic labeling methods are not compatible with peptides. Moreover, the presence of H-bond donors in peptides reduces the nucleophilicity of the fluoride to such degree that substitution reactions only proceed with very low yields. Thus, there is a great need for peptide labeling methods with a broad scope and amenable to simple implementation.

### SUMMARY

The present invention describes a method for ¹⁸F-labeling of a peptide supported on a solid-phase, said method comprising the following steps:
a. Providing a peptide protected with protecting groups and conjugated to a resin and coupled to Compound I:

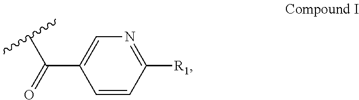

wherein R1 is an electron-withdrawing group.
b. Reacting the peptide of a. with [¹⁸F]fluoride.

Furthermore, the present invention also relates to a protected peptide conjugated to a resin, and coupled to [¹⁸F]6-fluoronicotinic acid.

### DESCRIPTION OF THE DRAWINGS

FIG. 1

Peptides of example 2 for radiolabelling.

FIG. 2

Radio-HPLC analysis of the labeling reaction of the peptides from FIG. 1. The radioactivity detector trace is superimposed with the UV trace of the ¹⁹F-standard. This confirms the chemical identity of the labelled peptides by comparison of the retention time in HPLC analysis with an authentic ¹⁹F synthesized standard. The UV trace shows the peak corresponding to the standard and the radio trace corresponds to the radioactivity detector in the UPLC in the same analysis.

FIG. 3

Radio-HPLC analysis of the labeling reaction of UCCB01-144 on solid-phase. The radioactivity detector trace is superimposed with the UV trace of the ¹⁹F-standard. This confirms the chemical identity of the labelled UCCB01-144 by comparison of the rettion time in HPLC analysis with an authentic ¹⁹F synthesized standard. The UV trace shows the peak corresponding to the standard and the radio trace corresponds to the radioactivity detector in the UPLC in the same analysis.

FIG. 4

Radio-HPLC analysis of the labeling reaction of UCCB01-144 in solution. The radioactivity detector traces are superimposed with the UV trace of the ¹⁹F-standard. The radio-HPLC analysis indicates that the entire ¹⁸F precursor is left unreacted.

FIG. 5

Radio-HPLC analysis of the labelling reaction of different valine pyridine precursors on solid-phase. The radioactivity detector traces are superimposed with the UV trace of the ¹⁹F-standard.

FIG. 6

Labeling and purification of [¹⁸F]Py-ACD model peptide. Radio-HPLC analysis of the crude reaction (black), overlaid with the UV tracer of the standard analysis (gray).

FIG. 7

Labeling and purification of [¹⁸F]Py-cRGDyK model peptide. Radio-HPLC analysis of the crude reaction (black), overlaid with the UV tracer of the standard analysis (gray).

### DETAILED DESCRIPTION OF THE INVENTION

Peptides allow targeting a vast range of biological targets including previously “undruggable” sites for small-molecule ligands. However, as mentioned above radiolabeling of peptides is difficult. This invention has utilised the single amino acid valine as a model, from which the inventors have provided a one-pot two-step procedure, wherein a resin-bound protected peptide can be efficiently radiolabelled with ¹⁸F. The reaction proceeds with more complex peptides ranging from the 5-mer opioid peptide YGGFL to the peptide-like drug UCCB01-144.

The present invention thus provides a simple, direct nucleophilic ¹⁸F-labeling strategy for radiolabeling peptides, which is universally applicable, scalable and reliable.

One aspect of the present invention relates to a method for ¹⁸F-labeling of a peptide, said method comprising the following steps:
a. Providing a peptide protected with protecting groups and conjugated to a resin and coupled to Compound I:

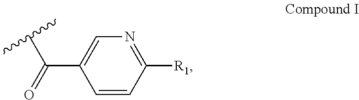

wherein R1 is an electron-withdrawing group.
b. Reacting the peptide of a. with [¹⁸F]fluoride.

This method enables a fast and highly efficient way of labeling peptides with [¹⁸F]fluoride.

The present invention further relates to a method for ¹⁸F-labeling of a peptide, said method comprising reacting a peptide, protected with protecting groups and conjugated to a resin and coupled to Compound I, with [¹⁸F]fluoride.

In one embodiment the method further comprises the following step:
c. Deprotecting the peptide.

As used herein, the term “deprotection” or “deprotecting” refers to a process by which a protective group is removed after the selective reaction is completed. Certain protective groups may be preferred over others due to their convenience or relative ease of removal. Without being limiting, deprotecting reagents for protected groups include strong acid such as trifluoroacetic acid (TFA), concentrated HCl, H₂SO₄, or HBr, and the like.

In a further embodiment the method further comprises the following step:
d. Cleaving the peptide from the resin.

In a preferred embodiment steps c. and d. are done simultaneously.

Furthermore, step c. and d. can be carried out immediately following step b. without an intermediate separation and purification of the product of step b. This gives the advantage of a faster total reaction time and maintains a higher degree of radioactivity in the final product.

In a further embodiment steps c. and d. are carried out under acidic conditions. In yet another embodiment steps c. and d. are carried out under TFA/TIPS/H2O or TFA/TIPS/H2O/DODT conditions, where TIPS is Triisopropylsilane and DODT is 2,2′-(Ethylenedioxy) diethanethiol.

In one embodiment, steps c. and d. are carried out under one or more of the following conditions: acidic, basic, reducing or oxidative conditions.

In one embodiment, steps c. and d. are carried out under acidic and/or reducing conditions.

The direct labeling strategy of the present invention has several advantages:
1) Since the labeling process is based on a one-pot, two-step procedure, simpler automatization is possible since no multi-step synthesizers are needed, which are more expensive and because of the additional required set-up more prone for errors.
2) The process is very fast and as such, more productions can be carried out in the same synthesizer. The method of the present invention for [¹⁸F]-labeling of a peptide takes between 30 min and 45 min including isolation and formulations.
3) Site-selectivity. The fluorine-18 is incorporated only at modified residue.

The total reaction time is crucial for [¹⁸F]-labeling due to the short half-life of [¹⁸F](109.8 min). The total reaction time of approximately 30-45 min of the direct labelling strategy of the present invention ensures that a high level of radioactivity is still remaining in the product after the radiolabelling is completed.

The necessary conditions for ensuring efficient radiolabeling of the peptide of the present invention such as high temperatures, strong basic environment or the need for organic aprotic solvents, would result in degradation of the peptide unless the amino acid side chains are protected by protecting groups. As the method of the present invention allows for the use of protecting groups, problems with degradation of the peptide due to harsh conditions can be circumvented. Additionally the method of the present invention also results in high yields of radiolabeling.

The term “protecting group” refers to a substituent that is commonly employed to block or protect a particular functionality while reacting with other functional group(s) on the compound. As used herein, “protecting group” refers to a moiety attached to a functional group to prevent an otherwise unwanted reaction of that functional group. Examples of functional groups include hydroxyl, amine, sulfhydryl, amide, carboxyl, carbonyl, etc.

In one embodiment of the method 80% of the side chains, such as 90%, such as 100% are protected by protecting groups. In a further embodiment the peptide is fully protected. Fully protected is to be understood as all side chains being coupled to protecting groups.

In another embodiment the protecting groups are acid-labile protecting groups.

The term “acid-labile protecting group” denotes protecting groups that are stable under basic conditions.

In a further embodiment the protecting groups are selected from the group consisting of: Alloc, Allyl, Nosyl, Mtt, ivDe.

In one embodiment the peptide contains at least two amino acid residues coupled through an amide bond. An amide bond is also commonly known as a peptide bond. In another embodiment the peptide contains at least three, four, six, nine or fourty amino acids coupled through amide bonds.

In a further embodiment the amino acids are natural amino acids also known as proteinogenic amino acids.

Solid-supported synthesis methods are highly advantageous for direct nucleophilic ¹⁸F-labeling strategy. They can easily be scaled and the solid-phase conjugated and protected precursor is amenable to the harsh conditions needed for direct nucleophilic fluorinations. Moreover, necessary precursor groups can be installed site-specifically at positions that are known not to disrupt target binding. As shown in example 3 of the present application solid phase radiolabeling of peptide UCCB01-144 results in decay-corrected radiochemistry conversion of 23%. On the other hand if radiolabeling of peptide UCCB01-144 is attempted using the same reaction conditions in solution no conversion is observed. Thus, solid-phase radiolabeling is necessary for efficient radiolabeling to occur.

For solid-phase radiolabeling the peptide of the present invention is conjugated to a resin.

The term “resin,” as used herein, refers to high molecular weight, insoluble polymer beads. Following examples are commonly used as resins for solid-phase synthesis of peptides: Polystyrene, polyacrylate, polyacrylamide, and polyethylene glycol resins.

In a preferred embodiment trityl-ChemMatrix resin is used. This resin displays a fast cleavage and high coupling efficacy.

Generally, resins in the range of 75 to 150 microns in diameter offer a good balance of reaction kinetics versus reliability.

In one embodiment the peptide is C-terminally conjugated to the resin.

The radiofluorination of step b. in the method of the present invention occurs via nucleophilic aromatic substitution at the pyridine moiety according to the reaction mechanism shown here below. Aromatic ¹⁸F-fluorination is attractive as the steric and electronic effects of substituting a hydrogen atom with fluorine are subtle, yet it can improve the metabolic stability of the parent compound.

Activated substrates can react with [¹⁸F]fluoride at room temperature, but with heating the reaction proceeds in the presence of hydrogen bond donors.

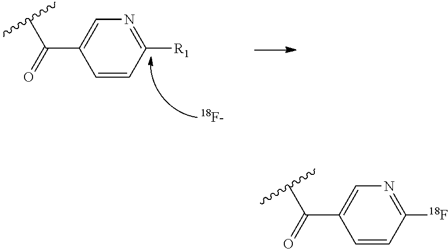

R₁ acts as a leaving group. For efficient nucleophilic substitution to occur, R₁ should be an electron withdrawing group.

The term “electron withdrawing group” refers to a chemical substituent that modifies the electrostatic forces acting on a nearby chemical reaction centre by withdrawing negative charge from that chemical reaction centre. Thus, electron withdrawing groups draw electrons away from a reaction centre. As a result, the reaction centre is fractionally more positive than it would be in the absence of the electron-withdrawing group. With increasing electronegativity of the leaving group the reaction rate for nucleophilic attack increases.

In one embodiment R₁ is selected from the group consisting of: trimethylammonium, phosphonium, sulfonium, nitro, iodonium salts or ylides, boronate esters and alkyltin groups (eg. SnMe₃ or SnBu₃). In a preferred embodiment R₁ is trimethylammonium.

Reaction time is a crucial parameter in radiofluorination due to the fast decay of [¹⁸F]fluoride. And since reaction temperature is a key regulator for increasing reaction time several reaction temperatures were tested.

In one embodiment the reaction temperature in step b. is at least 40° C., such as at least 60° C., such as at least 80° C.

As shown in table 1 of this application increasing the temperature form 40° C. to 60° C. increases the RCY from 2.6% to 21.5%. Furthermore, it can be seen in table 1 that a further increase in temperature from 60° C. to 80° C. results in an additional increase in RCY from 35.4% to 46.2%.

To ensure optimal conditions for the nucleophilic aromatic substitution it is generally preferred to use a polar aprotic solvent. In one embodiment the solvent in step b. is selected from the group consisting of DMSO, DMF, ACN, DMAc and NMP. It is to be understood that DMSO is dimethylsulfoxide, DMF is dimethylformamide and ACN is acetonitrile, DMAc is dimethylacetamide and NMP is N-Methyl-2-pyrrolidone.

The effect of different solvents on reaction time can be seen from the different conditions tested in table 1 of the present application. Changing the solvent from ACN to DMF results in an increase in RCY from 21.5% to 28.3%. The effect of changing the solvent from DMF to DMSO results in a further increase in RCY from 28.3% to 35.4%.

In a preferred embodiment the solvent in step b. is DMSO.

In one embodiment the reaction in step b. is in the presence of a phase-transfer catalyst. The phase-transfer catalyst can also be referred to as the activation salt.

In another embodiment the phase-transfer catalyst is selected from the group consisting of: TBA-OH, TBA/HCO₃, K₂₂₂/K₂CO₃, TBA-Tf, TBA-PO₃ and TBA-MS.

The full names of these complexes are as follows: TBA-OH is tetrabutylammonium-hydroxide, TBA/HCO₃ is tetrabutylammonium hydrogencarbonate/tetrabutylammonium bicarbonate, K₂₂₂/K2CO3 is 4,7,13, 16,21,24-Hexaoxa-1,10-diazabicyclo[8.8.8]hexacosane, Kryptofix 222/potassium carbonate, TBA-Tf is tetrabutylammonium triflate, TBA-PO³ is tetrabutylammonium phosphate and tetrabutylammonium mesylate is TBA-MS.

As can be seen from table 1 of the present application, the phase-transfer catalyst type has a significant impact on the RCY. Changing from K₂₂₂/K₂CO₃ to TBA-OH increases the RCY from 11.5% to 29.4%.

Thus, in a preferred embodiment the phase-transfer catalyst is TBA-OH or TBA-Tf

Another aspect of the present invention relates to a protected peptide conjugated to a resin, and coupled to [¹⁸F]6-fluoronicotinic acid.

A protected peptide is to be understood as a peptide protected with protecting groups.

In one embodiment the peptide is C-terminally coupled to the resin.

In one embodiment the [¹⁸F]6-fluoronicotinic acid is coupled to an —NH group according to the drawing below:

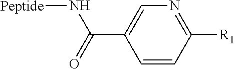

In a further embodiment the [¹⁸F]6-fluoronicotinic acid is coupled to the N-terminal —NH group.

In another embodiment the [¹⁸F]6-fluoronicotinic acid is coupled to an —NH group of Lysine.

### Examples

### Example 1—Radiolabeling of Valine Pyridine Precursor

A series of labeling experiments were carried out using a single amino acid representing the simplest model of a peptide-like structure.

Valine was conjugated to a trityl-ChemMatrix resin. This resin was chosen due to its fast cleavage and high coupling efficacy. Afterwards, valine was acylated at the N-terminus using a tetrafluorophenyl trimethylammoniumnicotinate ester (1). The conversion to NMe3Py-Valine (2) succeeded in >90% and ¹⁸F-labeling could be carried out in a next step (Table 1). Initial experiments were based on reported and optimized radiosynthesis of [¹⁸F]6-fluoronicotinic acid tetrafluorophenyl ester ([¹⁸F]F-Py-TFP) in solution using tetrabutylammonium bicarbonate (TBABC) in ACN at 40° C. In contrast to the reported radiochemical yield (RCY) of approximately 50% of [¹⁸F]F-Py-TFP, [¹⁸F]Py-Valine ([¹⁸F]3) could only be radiolabelled in a RCY of 2-3% on solid-phase. More than 60% of the initial added radioactivity was still resin-bound and could not be removed by washings with water, ethanol and dichloromethane, but >50% of the trapped radioactivity was eluted with TFA/TIPS-based cleavage solution. Radiochromatography analysis demonstrated that only 7% of peak area corresponded to [¹⁸F]3, whereas >90% corresponded to an unknown hydrophobic side-product. Solid-bound precursors without trimethylammonium groups resulted in substantial reduced trapping of the applied radioactivity and only the unknown hydrophobic side-product could be identified after the reaction and subsequent cleavage. These results suggest that the formed trimethylammonium resin (2) acts as an anion-exchanger, traps the [¹⁸F]fluoride with the trimethylammonium group and releases it as [¹⁸F]hydrofluoric acid or [¹⁸F]fluorotriisopropylsilane when the TFA/TIPS-based cleavage solution is added to the resin. Radio-HPLC analysis could indeed identify that this hydrophobic product is formed when the cleavage solution is mixed with aqueous [¹⁸F]fluoride. From this knowledge, it was hypothesized that the nucleophilic incorporation of [¹⁸F]fluoride could be increased at higher temperatures, by using reaction solvents such as DMSO known to promote the incorporation better than MeCN and by identifying a base facilitating best the incorporation (Table 1). Highest RCYs of approximately 50% were observed at 80° C. using DMSO and tetrabutylammonium hydroxide (TBA-OH). The reaction time was 10 min for all experiments presented in table 1.

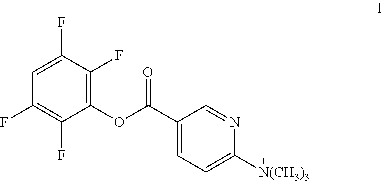

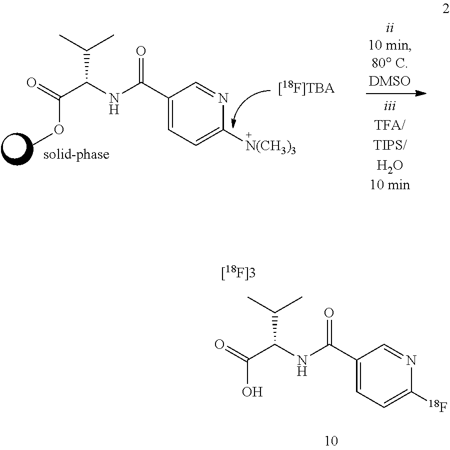

| | | | | Incorporation | | Radio- | |
|---|---|---|---|---|---|---|---|
| | | | | on solid- | Cleavage | HPLC | |
| | | | Temperature | phase | yield | RCC | RCY |
| Entry | PTC | Solvent | (° C.) | (%) | (%) | (%) | (%, ±SD)^{[a]} |
| 1 | TBA/HCO₃ | ACN | 40 | 67.6 | 51.4 | 7.4 | 2.6 |
| 2 | TBA/HCO₃ | ACN | 60 | 70.3 | 55.4 | 55.2 | 21.5 |
| 3 | TBA/HCO₃ | DMF | 60 | 61.2 | 62.4 | 74.0 | 28.3 |
| 4 | TBA/HCO₃ | DMSO | 60 | 75.8 | 61.0 | 76.8 | 35.4 |
| 5 | TBA/HCO₃ | DMSO | 80 | 83.2 | 60.8 | 91.3 | 46.2 |
| 6 | K₂₂₂/K₂CO₃ | DMSO | 80 | 47.5 | 67.1 | 36.0 | 11.5 |
| 7 | TBA-MS | DMSO | 80 | 61.4 | 65.8 | 43.2 | 17.5 |
| 8 | TBA-Tf | DMSO | 80 | 51.2 | 83.7 | 74.1 | 31.8 |
| 9 | TBA-OH | DMSO | 80 | 56.9 | 67.6 | 76.5 | 29.4 ± 17 |
| | | | | | | | (n = 4) |
| 10 | TBA-OH^{[b]} | DMSO | 80 | 76.6 | 70.2 | 90.6 | 48.9 ± 6 |
| | | | | | | | (n = 4) |

^{[a]}Pseudo radiochemical yields (pRCYs) were calculated considering the RadioHPLC RCC of the product, the cleavage yield and the incorporation yield. All values are decay-corrected
^{[b]}The [¹⁸F]TBA complex was dried at 85° C. In all the other entries the temperature was 110° C.
PTC: Phase-transfer catalyst

### Example 2—Labling of More Complex Peptides

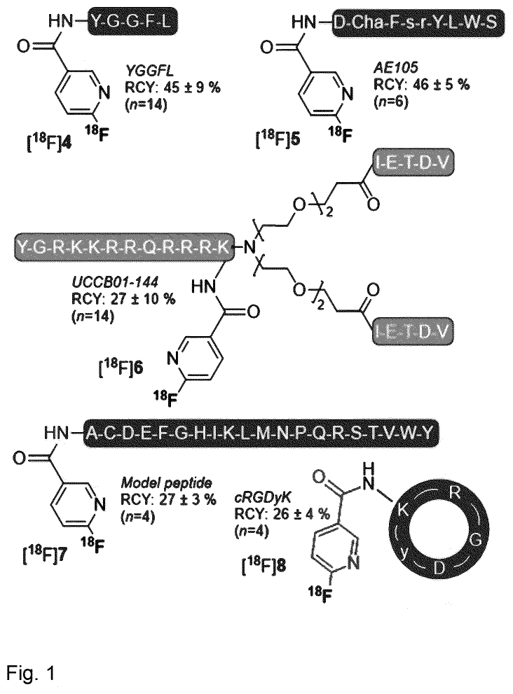

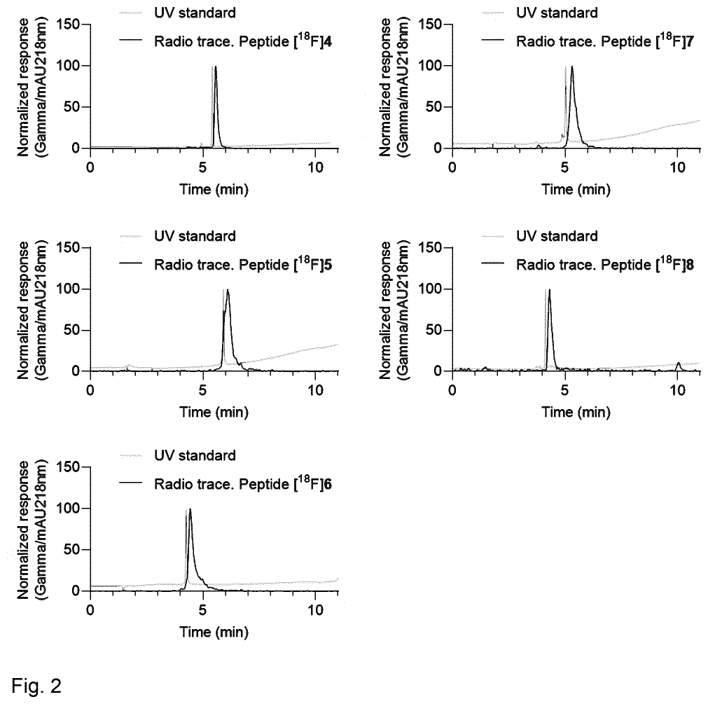

With an optimized method in hands, labeling of more complex peptides was carried out (FIG. 1). The first one was the 5-mer opioid peptide YGGFL (4). RCY of 41% was achieved and the conversions proceeded similarly to 2 despite the presence of the aromatic amino acids tyrosine and phenylalanine in 4. Next, the 9-mer peptide AE105 (5) was labelled. This peptide targets the urokinase-type plasminogen activator receptor and a ⁶⁸Ga-labelled AE105 tracer had promising results in a recent Phase I clinical trial. However, no fluorine-18 version of 5 is available yet. With the presented method, 5 was successfully labelled with almost 50% RCYs. The retention time of a synthesised 19F standard was compared with the retention time of the labelled product using the radio-detector trace to confirm that all peptides were correctly labelled and that the protection groups were cleaved (FIG. 2).

### Example 3—Labeling of UCCB01-144

### Solid-Phase Labeling of UCCB01-144

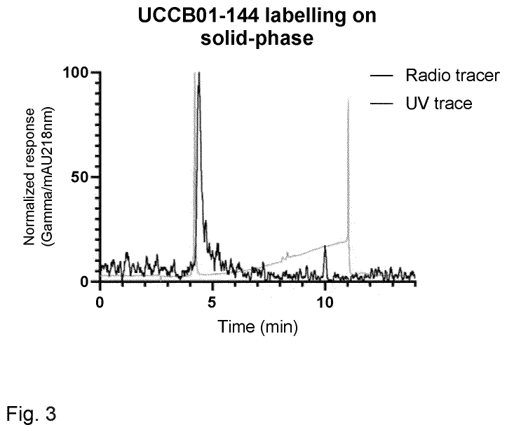

To assess if labeling was possible having the pyridinium synthon present at positions other than the N-terminus an analog version of the peptide UCCB01-144 (6 in FIG. 1) was synthesized. This analog is a potential neuroprotective peptide for ischemic stroke. An alloc-protected lysine was placed after the dimeric binding motif of 6, allowing the selective conjugation with 1. After the labelling, the syringe was drained, and the resin was washed with H₂O (2×1 mL), EtOH (2×1 mL) and DCM (2×1 mL). A mixture of TFA/TIPS/H2O (96:2:2) or TFA/TIPS/H2O/DODT (91/3/3/3) (1 mL) was aspirated into the syringe and cleaved for 10 min at 50° C. with magnetic stirring. The syringe was drained into an Eppendorf tube and the cleavage solution with radio-HPLC. UCCB01-144 (Scheme 1) was labelled with a decay-corrected radiochemistry conversion of 23% and the labelled product identity was confirmed by comparing the retention time of a ¹⁹F standard with the retention time of the product (FIG. 3, retention time: 4.4 min).

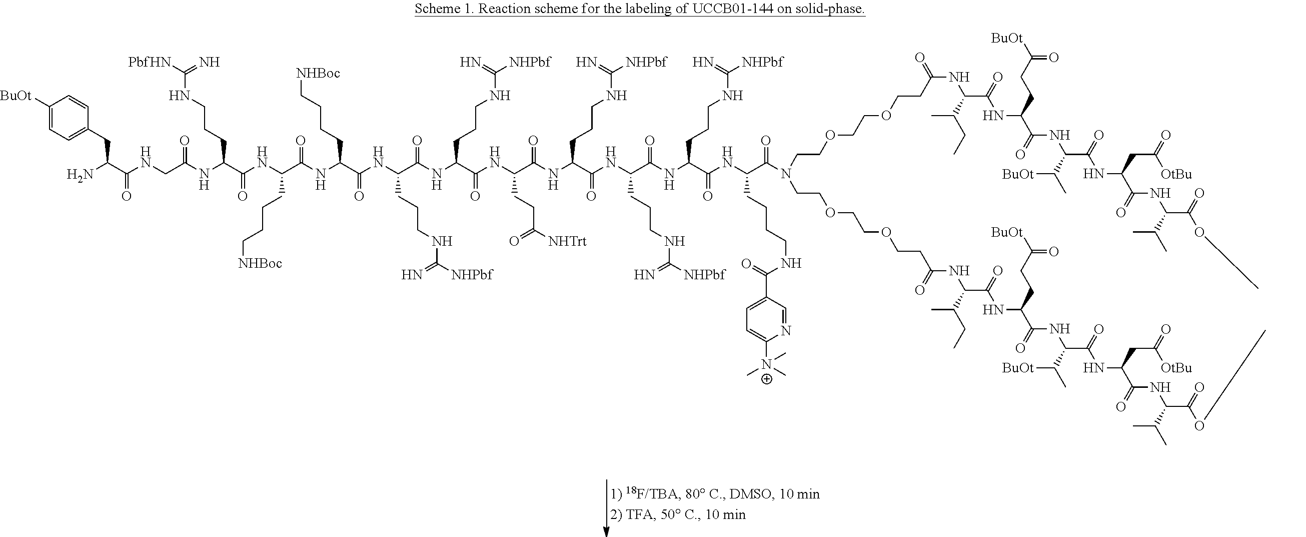

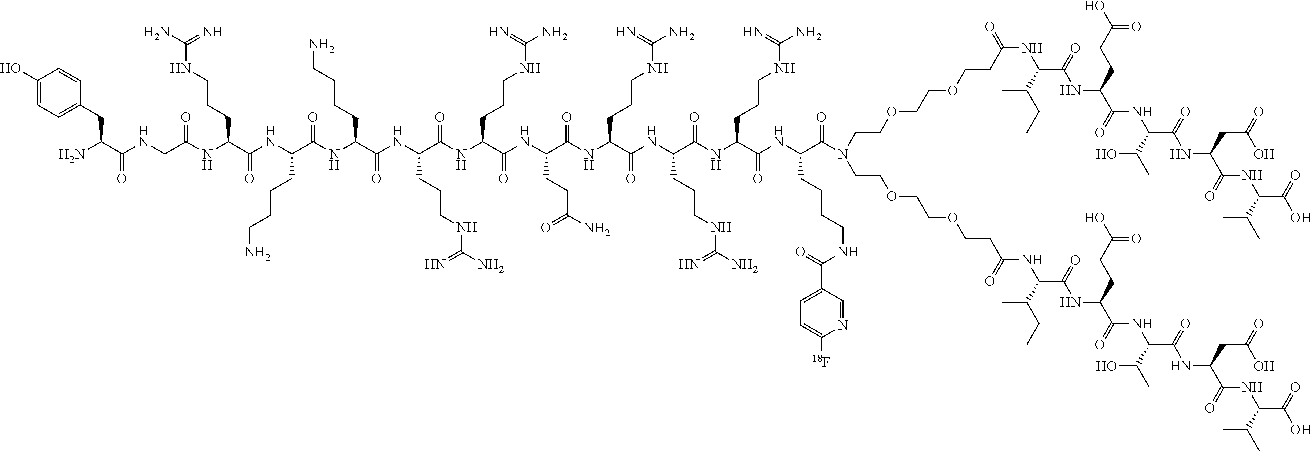 [SMILES: CCC(C)[C@H](NC(=O)CCOCCOCCN(CCOCCOCCC(=O)N[C@H](C(=O)N[C@@H](CCC(=O)O)C(=O)N[C@H](C(=O)N[C@@H](CC(=O)O)C(=O)N[C@H](C(=O)O)C(C)C)C(C)O)C(C)CC)C(=O)[C@H](CCCCNC(=O)c1ccc([18F])nc1)NC(=O)[C@H](CCCNC(=N)N)NC(=O)[C@H](CCCNC(=N)N)NC(=O)[C@H](CCCNC(=N)N)NC(=O)[C@H](CCC(N)=O)NC(=O)[C@H](CCCNC(=N)N)NC(=O)[C@H](CCCNC(=N)N)NC(=O)[C@H](CCCCN)NC(=O)[C@H](CCCCN)NC(=O)[C@H](CCCNC(=N)N)NC(=O)CNC(=O)[C@@H](N)Cc1ccc(O)cc1)C(=O)N[C@@H](CCC(=O)O)C(=O)N[C@H](C(=O)N[C@@H](CC(=O)O)C(=O)N[C@H](C(=O)O)C(C)C)C(C)O]

### Labeling of UCCB01-144 in Solution

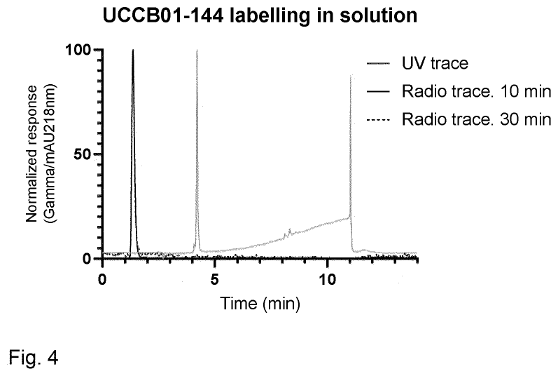

UCCB01-144 (Scheme 2) labeling in solution did not proceed up to 30 min of reaction time. The radio-HPLC analysis indicated that the entire ¹⁸F precursor is left unreacted (FIG. 4, retention time 1.3 min). Efficient labeling of UCCB01-144 requires not only the presence of the trimethylammonium synthon but also attachment of a fully protected peptide on a solid-support.

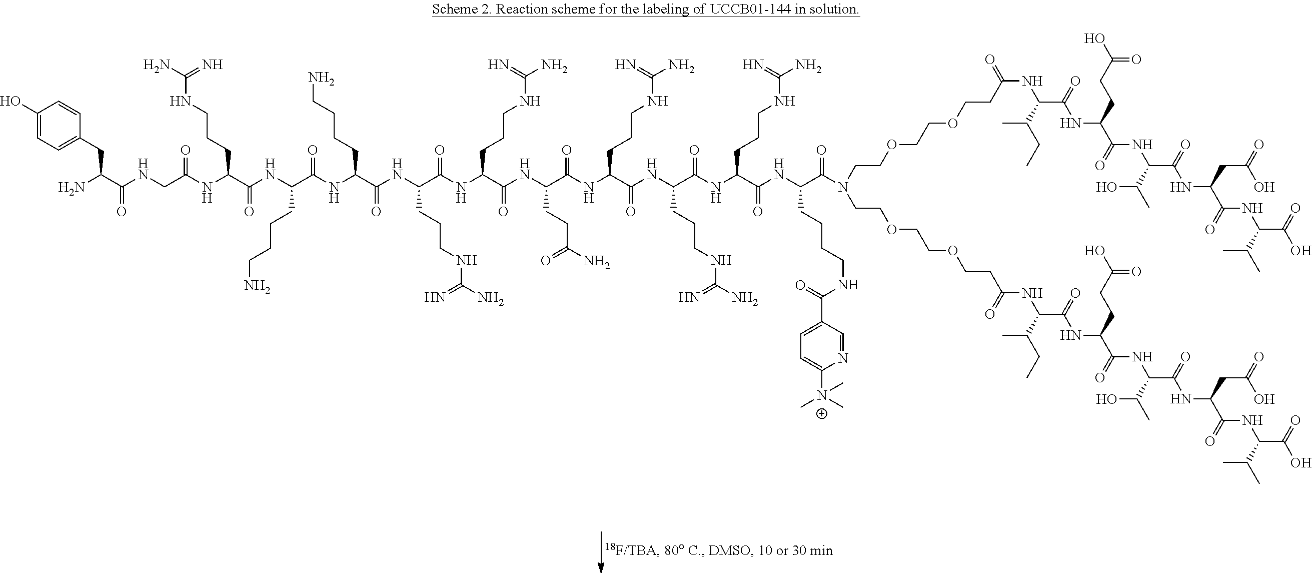 [SMILES: CCC(C)[C@H](NC(=O)CCOCCOCCN(CCOCCOCCC(=O)N[C@H](C(=O)N[C@@H](CCC(=O)O)C(=O)N[C@H](C(=O)N[C@@H](CC(=O)O)C(=O)N[C@H](C(=O)O)C(C)C)C(C)O)C(C)CC)C(=O)[C@H](CCCCNC(=O)c1ccc([N+](C)(C)C)nc1)NC(=O)[C@H](CCCNC(=N)N)NC(=O)[C@H](CCCNC(=N)N)NC(=O)[C@H](CCCNC(=N)N)NC(=O)[C@H](CCC(N)=O)NC(=O)[C@H](CCCNC(=N)N)NC(=O)[C@H](CCCNC(=N)N)NC(=O)[C@H](CCCCN)NC(=O)[C@H](CCCCN)NC(=O)[C@H](CCCNC(=N)N)NC(=O)CNC(=O)[C@@H](N)Cc1ccc(O)cc1)C(=O)N[C@@H](CCC(=O)O)C(=O)N[C@H](C(=O)N[C@@H](CC(=O)O)C(=O)N[C@H](C(=O)O)C(C)C)C(C)O]

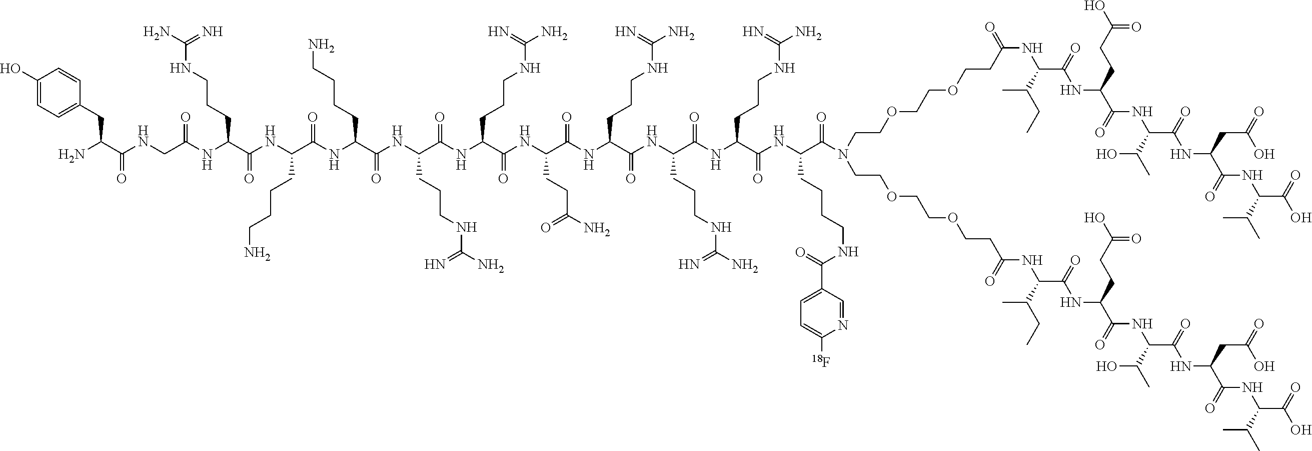 [SMILES: CCC(C)[C@H](NC(=O)CCOCCOCCN(CCOCCOCCC(=O)N[C@H](C(=O)N[C@@H](CCC(=O)O)C(=O)N[C@H](C(=O)N[C@@H](CC(=O)O)C(=O)N[C@H](C(=O)O)C(C)C)C(C)O)C(C)CC)C(=O)[C@H](CCCCNC(=O)c1ccc([18F])nc1)NC(=O)[C@H](CCCNC(=N)N)NC(=O)[C@H](CCCNC(=N)N)NC(=O)[C@H](CCCNC(=N)N)NC(=O)[C@H](CCC(N)=O)NC(=O)[C@H](CCCNC(=N)N)NC(=O)[C@H](CCCNC(=N)N)NC(=O)[C@H](CCCCN)NC(=O)[C@H](CCCCN)NC(=O)[C@H](CCCNC(=N)N)NC(=O)CNC(=O)[C@@H](N)Cc1ccc(O)cc1)C(=O)N[C@@H](CCC(=O)O)C(=O)N[C@H](C(=O)N[C@@H](CC(=O)O)C(=O)N[C@H](C(=O)O)C(C)C)C(C)O]

### Example 4—Solid-Phase Labelling of Valine Using Different Pyridine Precursors

The pyridine precursors containing either tetramethylammonium leaving group (1) or chloride (2) were synthesized and reacted with the dried ¹⁸F. As a control reaction, it was tested if any fluoride-18 is unspecifically incorporated to the solid-support when the pyridine moiety is absent (3).

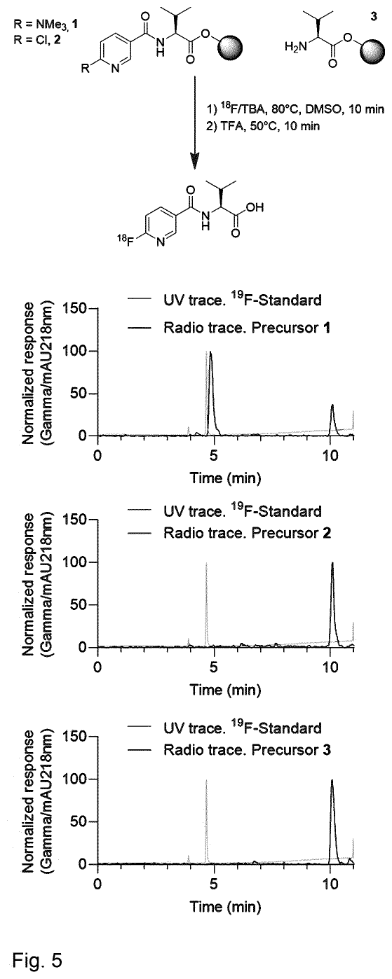

Labelling of precursor 1 resulted in 62% of radioactivity incorporation on the solid support and conversion to the expected product with a radiochromatography purity of 74%. The other two precursors resulted only in only 13% of assimilation on the solid support and no product was observed at the expected retention time of the ¹⁹F-valine standard, rt=4.7 min (FIG. 5).

### Example 5—Labeling of ACD Peptide

### Solid-Phase Labeling of ACD Model Peptide

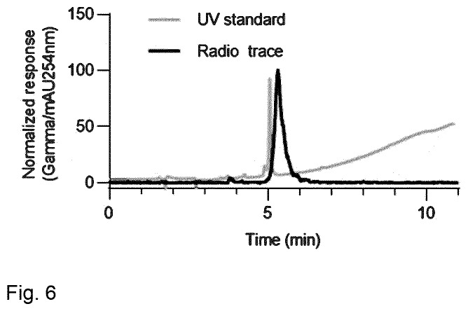

A model peptide containing the 20 common amino acids was prepared on solid-phase (7). Precursor 7 was labeled following the established procedure. After the labeling, the syringe was drained, and the resin was washed with H₂O (2×1 mL), EtOH (2×1 mL) and DCM (2×1 mL). A mixture of TFA/TIPS/H2O (96:2:2) or TFA/TIPS/H2O/DODT (91/3/3/3) (1 mL) was aspirated into the syringe and cleaved for 10 min at 50° C. with magnetic stirring. The syringe was drained the cleavage solution purified with radio-HPLC. ACD peptide (Scheme 3) was labeled with a decay-corrected radiochemistry conversion of 24% and the labeled product identity was confirmed by comparing the retention time of the labeled product with the retention time of a nonradioactive standard (FIG. 6, retention time: 5.3 min). This result illustrates that the method of the present invention is compatible with peptides containing combinations of all of the common amino acids.

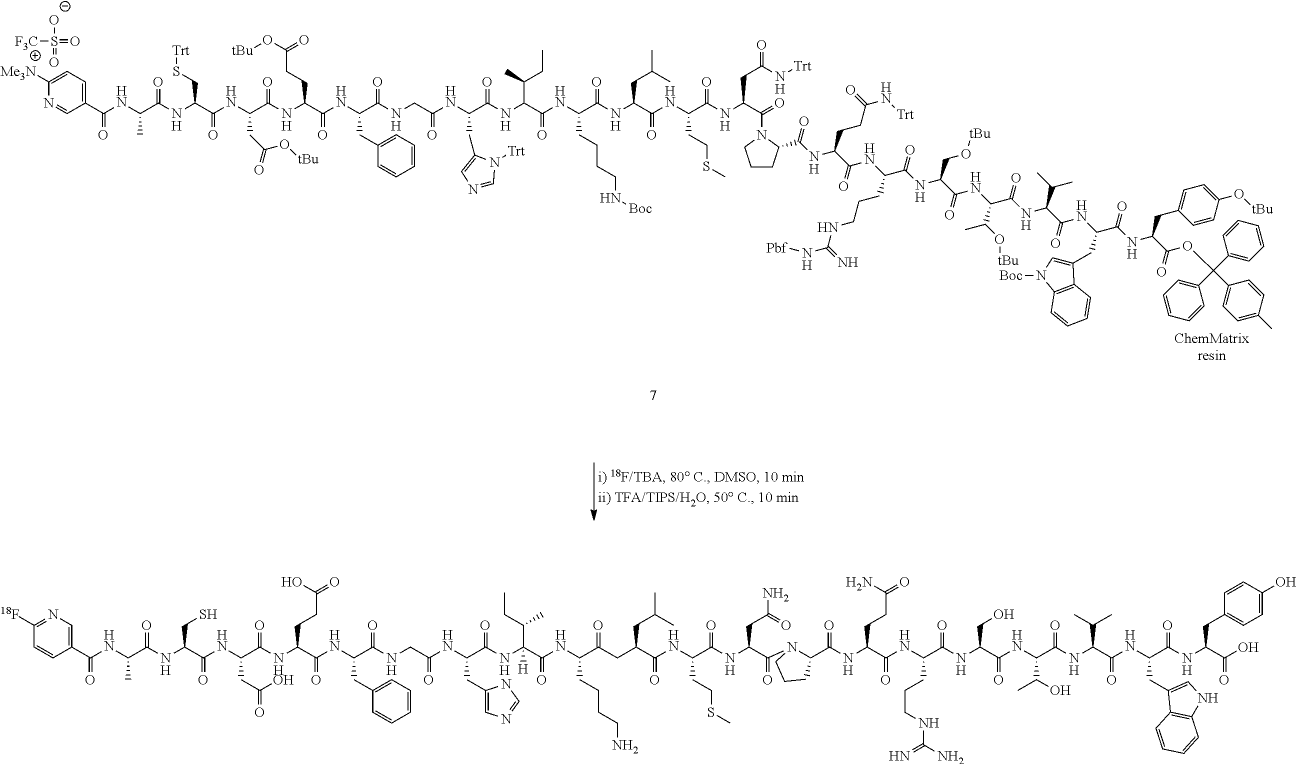

### Example 6—Labeling of cRGDyK Peptide

Solid-Phase Labeling of Cyclic Peptide cRGDyK

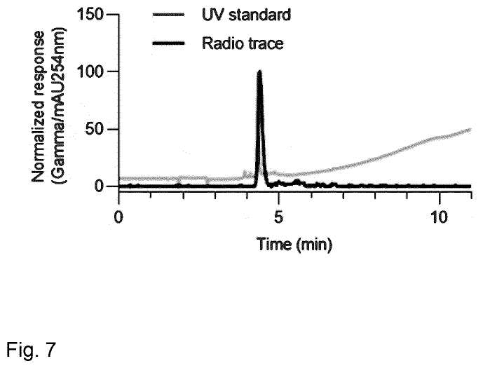

A model peptide containing the cyclic peptide RDGyK precursor was prepared on solid-phase (8). Precursor 8 was labeled following the established procedure. After the labeling, the syringe was drained, and the resin was washed with H₂O (2×1 mL), EtOH (2×1 mL) and DCM (2×1 mL). A mixture of TFA/TIPS/H2O (96:2:2) or TFA/TIPS/H2O/DODT (91/3/3/3) (1 mL) was aspirated into the syringe and cleaved for 10 min at 50° C. with magnetic stirring. The syringe was drained the cleavage solution purified with radio-HPLC. cRGDyK peptide (Scheme 4) was labeled with a decay-corrected radiochemistry conversion of 14% and the labeled product identity was confirmed by comparing the retention time of the labeled product with the retention time of a nonradioactive standard (FIG. 7, retention time: 4.4 min).

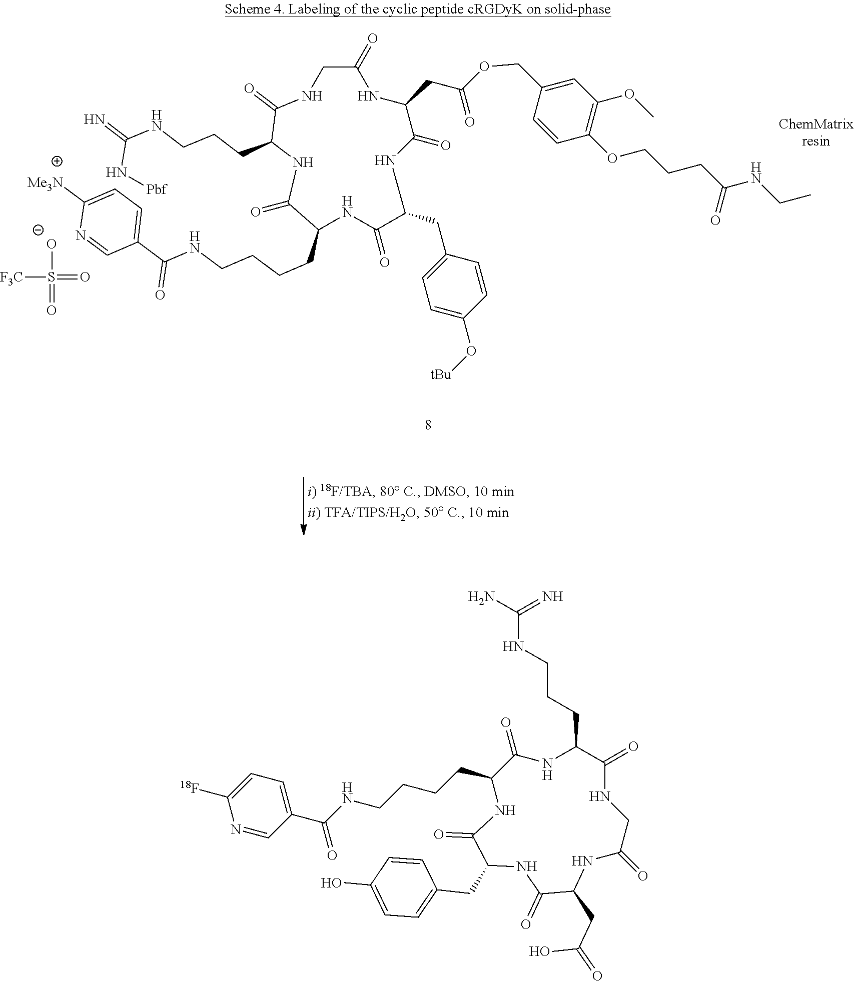

### Items

1. A method for ¹⁸F-labeling of a peptide, said method comprising the following steps:
a. Providing a peptide protected with protecting groups and conjugated to a resin and coupled to Compound I:

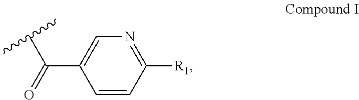

wherein R1 is an electron withdrawing group.
b. Reacting the peptide bound on a solid support as described in a. with [¹⁸F]fluoride.

2. The method according to item 1, further comprising the following step:
c. Deprotecting the peptide.

3. The method according to any of the preceding items, further comprising the following step:
d. Cleaving the peptide from the resin.

4. The method according to any of the preceding items, wherein steps c. and d. are done simultaneously.

5. The method according to any of the preceding items, wherein steps c. and d. are carried out under acidic conditions.

6. The method according to item 5, wherein steps c. and d. are carried out under TFA/TIPS/H2O conditions.

7. The method according to any of the preceding items, wherein the 80% of the side chains, such as 90%, such as 100% are protected by protecting groups.

8. The method according to any of the preceding items, wherein R1 is selected from the group consisting of trimethylammonium, phosphonium, sulfonium, nitro and iodonium.

9. The method according to any of the preceding items, wherein the reaction temperature in step b. is at least 40° C., such as at least 60° C., such as at least 80° C.

10. The method according to any of the preceding items, wherein the solvent in step b. is selected from the group consisting of DMSO, DMF and ACN.

11. The method according to any of the preceding items, wherein the reacting in step b. is in the presence of a phase-transfer catalyst.

12. The method according to item 11, wherein the phase-transfer catalyst is selected from the group consisting of: TBA-OH, TBA/HCO₃, K₂₂₂/K₂CO₃, TBA-Tf and TBA-PO₃ TBA-Ms.

13. A protected peptide conjugated to a resin, and coupled to [¹⁸F]6-fluoronicotinic acid.

14. The peptide according to item 13, wherein the peptide is C-terminally coupled to the resin.

15. The peptide according to any of items 13 and 14, wherein the [¹⁸F]6-fluoronicotinic acid is coupled to an —NH group.

16. The peptide according to any of the items 13-15, wherein the [¹⁸F]6-fluoronicotinic acid is coupled to the N-terminal-NH group.

17. The peptide according to any of the items 13-16, wherein the [¹⁸F]6-fluoronicotinic acid is coupled to an —NH group of Lysine or Arginine.

1. A method for ¹⁸F-labeling of a peptide, said method comprising the following steps:
a. Providing a peptide protected with protecting groups and conjugated to a resin and coupled to Compound I: 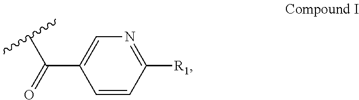
wherein R1 is an electron withdrawing group,
b. reacting the peptide bound on a solid support as described in a. with [¹⁸F]fluoride,
c. deprotecting the peptide, and
d. cleaving the peptide from the resin.

2. The method according to claim 1, wherein steps c. and d. are done simultaneously.

3. The method according to any of the preceding claims, wherein steps c. and d. are carried out under one or more of the following conditions: acidic, basic, reducing or oxidative conditions.

4. The method according to claim 3, wherein steps c. and d. are carried out under acidic and/or reducing conditions.

5. The method according to claim 4, wherein steps c. and d. are carried out under TFA/TIPS/H₂O conditions or under TFA/TIPS/H₂O/DODT conditions.

6. The method according to any of the preceding claims, wherein the 80% of the side chains, such as 90%, such as 100% are protected by protecting groups.

7. The method according to claim 6, wherein the protection groups are acid-labile protecting groups.

8. The method according to any of the preceding claims, wherein the protecting groups are selected from the group consisting of: tert-butoxycarbonyl (Boc), tert-Butyl (tBu) pentamethyl-2,3-dihydrobenzofuran-5-sulfonyl (Pbf), 4-Methyltrityl (Mtt), trityl (Trt), allyloxycarbonyl (aloc) or allyl (All), 9-fluorenylmethoxycarbonyl (Fmoc), 4-methoxy-2,3,6-trimethylphenylsulfonyl (Mtr).

9. The method according to any of the preceding claims, wherein the peptide is at least two amino acids coupled through an amide bond.

10. The method according to any of the preceding claims, wherein the peptide is at least three, four, six, nine or forty amino acids coupled through an amide bond.

11. The method according to any of the preceding claims, wherein the amino acids are natural amino acids.

12. The method according to any of the preceding claims, wherein peptide is C-terminally conjugated to the resin.

13. The method according to any of the preceding claims, wherein R1 is selected from the group consisting of trimethylammonium, phosphonium, sulfonium, nitro and iodonium.

14. The method according to claim 13, wherein R1 is trimethylammonium.

15. The method according to any of the preceding claims, wherein the reaction temperature in step b. is at least 40° C., such as at least 60° C., such as at least 80° C.

16. The method according to any of the preceding claims, wherein the solvent in step b. is selected from the group consisting of DMSO, DMF and ACN.

17. The method according to any of the preceding claims, wherein the reacting in step b. is in the presence of a phase-transfer catalyst.

18. The method according to claim 17, wherein the phase-transfer catalyst is selected from the group consisting of: TBA-OH, TBA/HCO₃, K₂₂₂/K₂CO₃, TBA-Tf and TBA-PO₃ TBA-Ms

19. The method according to claim 18, wherein the phase-transfer catalyst is TBA-OH or TBA-Tf


SEQUENCE LISTING (10 sequences)

SEQ ID NO: 1 - protein, 5 aa, Artificial
  synthetic peptide
  MISC_FEATURE (1)..(1): Labeled with precursor synthon
YGGFL

SEQ ID NO: 2 - protein, 5 aa, Artificial
  synthetic peptide
  MISC_FEATURE (1)..(1): Labeled with [18F]fluoronicotinic acid
YGGFL

SEQ ID NO: 3 - protein, 9 aa, Artificial
  synthetic peptide
  MISC_FEATURE (1)..(1): Labeled with precursor synthon
  MISC_FEATURE (2)..(2): X = Cha (3-cyclohexyl-L-alaine)
  MISC_FEATURE (4)..(4): D  amino acid
  MISC_FEATURE (5)..(5): D amino acid
DXFSRYLWS

SEQ ID NO: 4 - protein, 9 aa, Artificial
  synthetic peptide
  MISC_FEATURE (1)..(1): Labelled with [18F]fluoronicotinic acid
  MISC_FEATURE (2)..(2): X = Cha (3-cyclohexyl-L-alaine)
  MISC_FEATURE (4)..(4): D amino acid
  MISC_FEATURE (5)..(5): D amino acid
DXFSRYLWS

SEQ ID NO: 5 - protein, 12 aa, Artificial
  synthetic peptide
  MISC_FEATURE (12)..(12): Branch 1 - IETDV linked by PEG spacer
  MISC_FEATURE (12)..(12): Labeled with precursor synthon
  MOD_RES (12)..(12): AMIDATION
  MISC_FEATURE (12)..(12): Branch 2 - IETDV linked by PEG spacer
YGRKKRRQRRRK

SEQ ID NO: 6 - protein, 12 aa, Artificial
  synthetic peptide
  MISC_FEATURE (12)..(12): Branch 1 - IETDV linked by PEG spacer
  MOD_RES (12)..(12): AMIDATION
  MISC_FEATURE (12)..(12): Branch 2 - IETDV linked by PEG spacer
  MISC_FEATURE (12)..(12): Labeled with [18F]fluoronicotinic acid
YGRKKRRQRRRK

SEQ ID NO: 7 - protein, 20 aa, Artificial
  synthetic peptide
  MISC_FEATURE (1)..(1): Labeled with precursor synthon
ACDEFGHIKLMNPQRSTVWY

SEQ ID NO: 8 - protein, 20 aa, Artificial
  synthetic peptide
  MISC_FEATURE (1)..(1): Labeled with [18F]fluoronicotinic acid
ACDEFGHIKLMNPQRSTVWY

SEQ ID NO: 9 - protein, 5 aa, Artificial
  synthetic peptide
  MISC_FEATURE (1)..(5): cyclic peptide
  MISC_FEATURE (4)..(4): D amino acid
  MISC_FEATURE (5)..(5): Labeled with precursor synthon
RGDYK

SEQ ID NO: 10 - protein, 5 aa, Artificial
  synthetic peptide
  MISC_FEATURE (1)..(5): cyclic peptide
  MISC_FEATURE (4)..(4): D amino acid
  MISC_FEATURE (5)..(5): Labeled with [18F]fluoronicotinic acid
RGDYK